(19) United States
(12) Patent Application Publication
(10) Pub. No.: US 2026/0308083 A1
(43) Pub. Date: Oct. 8, 2026

(54) SELECTIVE OLEFIN OLIGOMERIZATION WITH TRANSITION METAL AMINE BIS(PHENOLATE) CATALYSTS

(71) Applicant: Chevron Phillips Chemical Company LP, The Woodlands, TX (US)

(72) Inventors: Thilina Gunasekara, Bartlesville, OK (US); Qing Yang, Bartlesville, OK (US)

(21) Appl. No.: 19/632,543

(22) Filed: Mar. 30, 2026

Related U.S. Application Data
(60) Provisional application No. 63/785,149, filed on Apr. 8, 2025.

(51) Int. Cl. C07C 2/32; B01J 31/18; B01J 31/22; C07C 2/30

(52) U.S. Cl. CPC C07C 2/32; B01J 31/1835; B01J 31/2213; B01J 31/2217; B01J 31/2295; C07C 2/30; B01J 2231/20; B01J 2531/004; B01J 2531/0241; B01J 2531/48; C07C 2531/14; C07C 2531/18; C07C 2531/22

(57) ABSTRACT

Catalyst compositions comprising transition metal amine bis(phenolate) complexes and activator-supports are disclosed herein for their use in olefin oligomerization processes to produce oligomer products. Oligomerization processes employing the catalyst compositions selectively produce a mixture of olefin dimers and trimers, wherein the dimers almost exclusively contain vinylidene end groups. Oligomer products disclosed herein can exclude higher order oligomer products, such as tetramers and above.

### REFERENCE TO RELATED APPLICATION

This application claims the benefit of U.S. Provisional Patent Application No. 63/785,149, filed on Apr. 8, 2025, the disclosure of which is incorporated herein by reference in its entirety.

### TECHNICAL FIELD

The present disclosure relates generally to catalyst compositions comprising transition metal amine bis(phenolate) complexes and activator-supports, and their use in oligomerization processes.

### BACKGROUND

Suitable catalysts that can selectively produce olefin dimers and trimers are highly desirable. The presence of vinylidene end groups in dimer products is desired to facilitate further reaction of the dimer to trimers and higher oligomers.

Transition metal amine bis(phenolate) complexes have been disclosed for use in olefin polymerization reactions as part of a heterogenous catalyst system. However, these catalysts have not previously been applicable to the production of oligomer products. Transition metal amine bis(phenolate) complexes can produce higher molecular weight products, and the presence of activator-supports within a catalyst composition can produce multiple undesirable oligomer isomers. Therefore, catalyst compositions comprising transition metal amine bis(phenolate) complexes that selectively yield dimer and trimer oligomer products, particularly dimer products containing vinylidene end groups, are desired, and it is to these ends that the present disclosure is generally directed.

### SUMMARY

This summary is provided to introduce a selection of concepts in a simplified form that are further described herein. This summary is not intended to identify required or essential features of the claimed subject matter. Nor is this summary intended to be used to limit the scope of the claimed subject matter.

Catalyst compositions disclosed herein can comprise a catalyst having Formula (I).

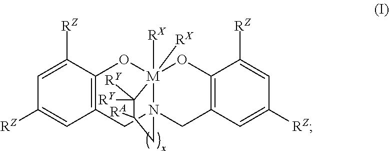

and an activator-support. In Formula (I), M can be Zr or Hf. Each R^{X} independently can be a halogen or a C₁ to C₁₈ hydrocarbyl group, each R independently can be a C₂ to C₁₂ hydrocarbyl group, each R^{Z} independently can be H or a C₁ to C₁₂ hydrocarbyl group, and x can be 1 or 2. In other aspects, x can be 3 or more. In certain aspects, R^{A} can be H or joined with an R^{Y} group to form an aromatic heterocyclic group.

Processes to prepare oligomerization catalyst compositions are also disclosed herein, and can comprise (i) contacting an activator-support and an organoaluminum compound to form a first mixture, and (ii) contacting the first mixture and a transition metal amine bis(phenolate) complex to form the catalyst composition. Catalyst compositions prepared in this manner can be used in oligomerization processes which can comprise (i) contacting the catalyst composition, a C₄ to C₃₀ alpha olefin monomer, and optionally hydrogen, in an oligomerization reactor under oligomerization conditions, and (ii) forming an oligomer product in the oligomerization reactor.

Both the foregoing summary and the following detailed description provide examples and are explanatory only. Accordingly, the foregoing summary and the following detailed description should not be considered to be restrictive. Further, features or variations may be provided in addition to those set forth herein. For example, certain aspects may be directed to various feature combinations and sub-combinations described in the detailed description.

### BRIEF DESCRIPTION OF THE FIGURES

The following figures form part of the present specification and is included to further demonstrate certain aspects of the present invention. The invention may be better understood by reference to these figures in combination with the detailed description.

FIG. 1 presents a chromatograph of the oligomer product formed in Example 1.

FIG. 2 presents a chromatograph of the oligomer product formed in Example 4.

While the inventions disclosed herein are susceptible to various modifications and alternative forms, only a few specific aspects have been shown by way of example in the drawings and described in detail below. The figures and detailed description of specific aspects are not intended to limit the breadth or scope of the inventive concepts or the appended claims in any manner. Rather, the figures and detailed description are provided to illustrate the inventive concepts to a person of ordinary skill in the art and to enable such person to make and use the inventive concepts.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Herein, features of the subject matter are described such that, within particular aspects, a combination of different features can be envisioned. For each and every aspect and each and every feature disclosed herein, all combinations that do not detrimentally affect the compounds, compositions, processes, or methods described herein are contemplated with or without explicit description of the particular combination. Additionally, unless explicitly recited otherwise, any aspect or feature disclosed herein can be combined to describe inventive compounds, compositions, processes, or methods consistent with the present disclosure.

In this disclosure, while compositions and processes/methods are described in terms of “comprising” various materials or components and steps, the compositions and processes/methods also can “consist essentially of” or “consist of” the various materials or components and steps, unless stated otherwise. For example, a catalyst composition consistent with aspects of the present invention can comprise; alternatively, can consist essentially of; or alternatively, can consist of; a transition metal amine bis(phenolate) compound, an organoaluminum compound, and an activator-support. The terms “a,” “an,” and “the” are intended to include plural alternatives, e.g., at least one, unless otherwise specified. For instance, the disclosure of “an organoaluminum compound” is meant to encompass one, or mixtures or combinations of two or more, organoaluminum compound(s), unless otherwise specified.

For any generic or specific compound or group disclosed herein, any name or structure presented is intended to encompass all conformational isomers, regioisomers, stereoisomers, and mixtures thereof that can arise from a particular set of substituents, unless otherwise specified. The name or structure also encompasses all enantiomers, diastereomers, and other optical isomers (if there are any), whether in enantiomeric or racemic forms, as well as mixtures of stereoisomers, as would be recognized by a skilled artisan, unless otherwise specified. For example, a general reference to hexene (or hexenes) includes all linear or branched, acyclic or cyclic, hydrocarbon compounds having six carbon atoms and 1 carbon-carbon double bond; a general reference to pentane includes n-pentane, 2-methyl-butane, and 2,2-dimethylpropane; and a general reference to a butyl group includes an n-butyl group, a sec-butyl group, an iso-butyl group, and a t-butyl group.

The terms “contacting” and “combining” are used herein to describe compositions and processes/methods in which the materials are contacted or combined together in any order, in any manner, and for any length of time, unless otherwise specified. For example, the materials can be blended, mixed, slurried, dissolved, reacted, treated, impregnated, compounded, or otherwise contacted or combined in some other manner or by any suitable method or technique.

The term “hydrocarbon” whenever used in this specification and claims refers to a compound containing only carbon and hydrogen, whether saturated or unsaturated. Other identifiers can be utilized to indicate the presence of particular groups in the hydrocarbon (e.g., halogenated hydrocarbon indicates that the presence of one or more halogen atoms replacing an equivalent number of hydrogen atoms in the hydrocarbon). The term “hydrocarbyl group” is used herein in accordance with the definition specified by IUPAC: a univalent group formed by removing a hydrogen atom from a hydrocarbon (that is, a group containing only carbon and hydrogen). Non-limiting examples of hydrocarbyl groups include alkyl, alkenyl, aryl, and aralkyl groups, amongst other groups.

The term “olefin” includes aliphatic and aromatic, cyclic and acyclic, and/or linear and branched compounds having at least one carbon-carbon double bond that is not part of an aromatic ring or ring system, unless specifically stated otherwise. Olefins having only one, only two, only three, etc., carbon-carbon double bonds can be identified by use of the term “mono,” “di,” “tri,” etc., within the name of the olefin. Olefins can be further identified by the position of the carbon-carbon double bond(s). The term “alpha olefin” as used herein refers to any olefin that has a double bond between the first and second carbon atom of a contiguous chain of carbon atoms.

The term “substituted” when used to describe a group, for example, when referring to a substituted analog of a particular group, is intended to describe any non-hydrogen moiety that formally replaces a hydrogen in that group, and is intended to be non-limiting. A group or groups can also be referred to herein as “unsubstituted” or by equivalent terms such as “non-substituted,” which refers to the original group in which a non-hydrogen moiety does not replace a hydrogen within that group. Unless otherwise specified, “substituted” is intended to be non-limiting and include inorganic substituents or organic substituents as understood by one of ordinary skill in the art.

The “activator-support” of this disclosure can be a chemically-treated solid oxide. The terms “support” and “activator-support” are not used to imply these components are inert, and such components should not be construed as an inert component of the catalyst composition. The term “activator,” as used herein, refers generally to a substance that is capable of converting a transition metal amine bis(phenolate) compound into an active catalyst that can oligomerize olefins. This term is used regardless of the actual activating mechanism. Aluminoxanes, organoboron or organoborate compounds, and ionizing ionic compounds generally are referred to as co-catalysts herein.

The term “transition metal amine bis(phenolate) compound” as used herein describes compounds comprising a transition metal coordinated to an amine bis(phenolate) ligand. In some contexts, the transition metal amine bis(phenolate) compound is referred to simply as the “catalyst,” in much the same way the term “co-catalyst” is used herein to refer to, for example, an organoaluminum compound.

The terms “catalyst composition,” “catalyst mixture,” “catalyst system,” and the like, do not depend upon the actual product or composition resulting from the contact or reaction of the initial components of the disclosed or claimed catalyst composition (or catalyst mixture or catalyst system), the nature of the activated catalyst composition, or the fate of the activator, the organoaluminum compound, and the transition metal amine bis(phenolate) compound after combining these components. Therefore, the terms “catalyst composition,” “catalyst mixture,” “catalyst system,” and the like, encompass the initial starting components of the composition, as well as whatever product(s) may result from contacting these initial starting components. The terms “catalyst composition,” “catalyst mixture,” “catalyst system,” and the like, may be used interchangeably throughout this disclosure.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein. For example, the weight ratio of the transition metal amine bis(phenolate) compound to the activator-support in the catalyst composition can be in various ranges. By a disclosure that the weight ratio can be in a range from 1:1 to 1:1000, the intent is to recite that the weight ratio can be any weight ratio within the range and, for example, can include any range or combination of ranges from 1:1 to 1:1000, such as from 1:1 to 1:500, from 1:10 to 1:350, or from 1:50 to 1:200, and so forth. Likewise, all other ranges disclosed herein should be interpreted in a manner similar to this example.

In general, an amount, size, formulation, parameter, range, or other quantity or characteristic is “about” or “approximate” whether or not expressly stated to be such. Whether or not modified by the term “about” or “approximately,” the claims include equivalents to the quantities or characteristics.

Although any methods and materials similar or equivalent to those described herein can be used in the practice or testing of the invention, the typical methods and materials are herein described.

All publications and patents mentioned herein are incorporated herein by reference in their entirety for the purpose of describing and disclosing, for example, the constructs and methodologies that are described in the publications and patents, which might be used in connection with the presently described invention.

### DETAILED DESCRIPTION

Disclosed herein are catalyst compositions containing transition metal amine bis(phenolate) complexes and an activator-support, and olefin oligomerization processes utilizing the catalyst compositions to selectively produce olefin dimers and trimers.

### Catalyst Compositions

In accordance with aspects of the present invention, catalyst compositions are disclosed herein comprising a transition metal amine bis(phenolate) complex and an activator-support. In certain aspects, these catalyst compositions can further comprise an organoaluminum compound. The transition metal amine bis(phenolate) complex can be a compound having Formula (I):

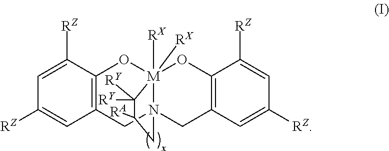

Within Formula (I), M, x, R^{A}, R^{X}, R^{Y}, and each R^{Z} are independent elements of the transition metal amine bis(phenolate) complex. Unless otherwise specified, Formula (I) above, any other structural formulas disclosed herein, and any transition metal amine bis(phenolate) complex, compound, or species disclosed herein are not designed to show stereochemistry or isomeric positioning of the different moieties (e.g., these formulas are not intended to display cis or trans isomers, or R or S diastereoisomers), although such compounds are contemplated and encompassed by these formulas and/or structures.

In accordance with aspects of this invention, M in Formula (I) can be Zr or Hf. Thus, M can be Zr in one aspect, and M can be Hf in another aspect. In Formula (I), each R^{X} independently can be a halide (e.g., F, Cl, Br, etc.) or a C₁ to C₁₈ hydrocarbyl group; or alternatively, a C₁ to C₈ hydrocarbyl group. Hydrocarbyl is used herein to specify a hydrocarbon radical group and includes, for instance, aryl, alkyl, cycloalkyl, alkenyl, cycloalkenyl, cycloalkadienyl, alkynyl, aralkyl, aralkenyl, and aralkynyl groups.

For example, each R^{X} independently can be a methyl group, an ethyl group, a propyl group, a butyl group (e.g., t-Bu), a pentyl group, a hexyl group, a heptyl group, an octyl group, a nonyl group, a decyl group, an ethenyl group, a propenyl group, a butenyl group, a pentenyl group, a hexenyl group, a heptenyl group, an octenyl group, a nonenyl group, a decenyl group, a phenyl group, a tolyl group, a benzyl group, or a naphthyl group, and the like, in certain aspects of the present invention. In other aspects, each R^{X} independently can be a methyl group, an ethyl group, a propyl group, a butyl group, a pentyl group, a hexyl group, a phenyl group, or a benzyl group. In certain aspects, each R^{X} can be the same, thus, each R^{X} can be a methyl group; alternatively, each R^{X} can be a phenyl group; or alternatively, each R^{X} can be a benzyl group.

Each R^{Y} independently can be any C₂ to C₁₂ hydrocarbyl group; or alternatively, a C₂ to C₈ hydrocarbyl group. Thus, each R^{Y} independently can be an ethyl group, a propyl group, a butyl group (e.g., t-Bu), a pentyl group, a hexyl group, a heptyl group, an octyl group, a nonyl group, a decyl group, an ethenyl group, a propenyl group, a butenyl group, a pentenyl group, a hexenyl group, a heptenyl group, an octenyl group, a nonenyl group, a decenyl group, a phenyl group, a tolyl group, a benzyl group, or a naphthyl group, and the like. In certain aspects, each R^{Y} can be the different, thus, one R can be a propyl group and another R^{Y} can be an ethyl group. In other aspects, each R^{Y} can be the same, thus, each R^{Y} can be an ethyl group; alternatively, each R^{Y} can be a propyl group; or alternatively, each R^{Y} can be a butyl group.

Similarly, each R^{Z} can be H or a C₁ to C₁₂ hydrocarbyl group. As for R^{X}, each R^{Z} independently can be a methyl group, an ethyl group, a propyl group, a butyl group (e.g., t-Bu), a pentyl group, a hexyl group, a heptyl group, an octyl group, a nonyl group, a decyl group, an ethenyl group, a propenyl group, a butenyl group, a pentenyl group, a hexenyl group, a heptenyl group, an octenyl group, a nonenyl group, a decenyl group, a phenyl group, a tolyl group, a benzyl group, or a naphthyl group, and the like. In one aspect, each R^{Z} can be the same, while in another aspect, each R^{Z} can be different. For instance, each R^{Z} can be H; alternatively, a methyl group; or alternatively, a butyl group (e.g., tert-butyl).

An illustrative and non-limiting example of an amine bis(phenolate) complex having formula (I) can include the following compound (Et=ethyl, Bn=benzyl; tBu=tert-butyl):

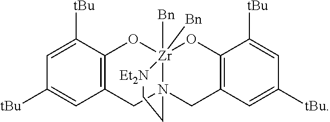

Formula (I) shows an ONNO amine bis(phenolate) ligand coordinated to the metal, comprising a hydrocarbyl linking group between the two nitrogen atoms coordinated to the metal. The linking group is defined by a length of two carbons, sufficient to bridge the three-dimensional distance required for each amine to coordinate to the metal atom. In certain aspects, the length of the linking group may be greater than 2, as denoted by the variable “x” shown in Formula (I). In certain aspects, x can be 1, 2, 3, or more.

For instance, further examples of transition metal amine bis(phenolate) complexes having larger or more elaborate structures connecting the phenolate moieties to the coordinated metal are described in U.S. Pat. No. 9,663,594, and also contemplated as suitable for the catalyst compositions disclosed herein. Certain ligands disclosed therein are shown as comprising a heterocycle (e.g., pyridine) between the phenolate moieties. Such examples may further comprise a pendant amine as a linking group to coordinate to the metal as an ONNO ligand. However, linker length x in such examples may be in a range from 3 to 6 carbon atoms, e.g., 3, 4, 5, or 6 carbon atoms, or more.

The linking group defined by the hydrocarbyl chain of length x can be substituted in certain aspects, as represented by R^{A} in Formula (I). Where the linking group is unsubstituted, R^{A} can be H. Alternatively, R^{A} can be joined with a R^{Y} to form a heterocyclic group containing the coordinated nitrogen. In certain aspects, the heterocyclic group thus formed can be a substituted or unsubstituted pyrrole, a substituted or unsubstituted pyrrolidine, a substituted or unsubstituted piperidine, or a substituted or unsubstituted pyridine.

In addition to transition metal amine bis(phenolate) compounds described above, catalyst compositions of the present invention can comprise an organoaluminum compound (e.g., an alkylaluminum compound). In some aspects, the organoaluminum compound can be a trialkylaluminum, an alkylaluminum alkoxide, an alkylaluminum halide, or any combination thereof. In other aspects, the organoaluminum compound can be a trialkylaluminum; alternatively, an alkylaluminum halide; or alternatively, an alkylaluminum alkoxide.

Each alkyl group of an alkylaluminum compound independently can be a C₁ to C₂₀ alkyl group; alternatively, a C₁ to C₁₀ alkyl group; or alternatively, a C₁ to C₆ alkyl group. In an aspect, each alkyl group of an alkylaluminum compound independently can be a methyl group, an ethyl group, a propyl group, a butyl group, a pentyl group, a hexyl group, a heptyl group, or an octyl group; or alternatively, a methyl group, an ethyl group, a butyl group, a hexyl group, or an octyl group. In some aspects, each alkyl group of an alkylaluminum compound can be a methyl group, an ethyl group, an n-propyl group, an n-butyl group, an iso-butyl group, an n-hexyl group, or an n-octyl group; alternatively, a methyl group, an ethyl group, an n-butyl group, or an iso-butyl group; alternatively, a methyl group; alternatively, an ethyl group; alternatively, an n-propyl group; alternatively, an n-butyl group; alternatively, an iso-butyl group; alternatively, an n-hexyl group; or alternatively, an n-octyl group.

While not being limited thereto, representative organoaluminum compounds can include trimethylaluminum, triethylaluminum, tri-n-propylaluminum, tri-n-butylaluminum, triisobutylaluminum, tri-n-hexylaluminum, tri-n-octylaluminum, diisobutylaluminum hydride, diethylaluminum ethoxide, diethylaluminum chloride, and the like, as well as any combination thereof.

Catalyst compositions disclosed herein can comprise an activator-support, for example, an activator-support comprising a solid oxide treated with an electron-withdrawing anion. For instance, the activator-support can comprise fluorided alumina, chlorided alumina, bromided alumina, sulfated alumina, fluorided silica-alumina, chlorided silica-alumina, bromided silica-alumina, sulfated silica-alumina, fluorided silica-zirconia, chlorided silica-zirconia, bromided silica-zirconia, sulfated silica-zirconia, fluorided silica-titania, fluorided-chlorided silica-coated alumina, fluorided silica-coated alumina, sulfated silica-coated alumina, or phosphated silica-coated alumina, and the like, as well as any combination thereof. In some aspects, the activator-support can comprise a fluorided solid oxide and/or a sulfated solid oxide, while in other aspects, the activator-support can comprise fluorided silica-coated alumina, sulfated alumina, or a combination thereof. Examples of such materials are disclosed in, for instance, U.S. Pat. Nos. 7,294,599, 7,601,665, 7,884,163, 8,309,485, 8,623,973, and 9,023,959.

Catalyst compositions disclosed herein also can comprise additional cocatalysts, such as an aluminoxane compound, an organoboron or organoborate compound, an ionizing ionic compound, and the like, or any combination thereof. In an aspect, the catalyst composition can comprise an aluminoxane compound. The aluminoxane compound can comprise, consist essentially of, or consist of, methylaluminoxane (MAO), ethylaluminoxane, modified methylaluminoxane (MMAO), n-propylaluminoxane, iso-propyl-aluminoxane, n-butylaluminoxane, sec-butylaluminoxane, iso-butylaluminoxane, t-butylaluminoxane, 1-pentylaluminoxane, 2-pentylaluminoxane, 3-pentyl-aluminoxane, iso-pentyl-aluminoxane, neopentylaluminoxane, or mixtures thereof. In yet another aspect, the catalyst composition can comprise an organoboron or organoborate compound. In still another aspect, the catalyst composition can comprise an ionizing ionic compound. Alternatively, the catalyst composition may be substantially free of aluminoxane compounds, organoboron and organoborate compounds, ionizing ionic compounds, or combinations thereof.

Components of the catalyst composition disclosed herein can be present in any suitable relative amount. For example, the weight ratio of the transition metal amine bis(phenolate) compound to the activator-support in the catalyst composition is not particularly limited. Nonetheless, this weight ratio often falls within a range from 1:1 to 1:1000, such as from 1:1 to 1:500, from 1:10 to 1:350, or from 1:50 to 1:200. Likewise, the weight ratio of the organoaluminum compound to the activator-support in the catalyst composition is not particularly limited. Illustrative and non-limiting ranges of this weight ratio include from 10:1 to 1:100, from 10:1 to 1:10, or from 1:1 to 1:10.

In other aspects, the amount of the organoaluminum compound in the catalyst composition can be measured relative to the amount of the transition metal amine bis(phenolate) compound. Weight ratios of the organoaluminum to the transition metal amine bis(phenolate) compound in the catalyst compositions disclosed herein can range from 100:1 to 1:100, from 100:1 to 1:10, or from 50:1 to 1:1. If, for example, more than one transition metal amine bis(phenolate) compound and/or more than one organoaluminum are employed, these ratios are based on the total moles of respective transition metals and organoaluminums.

Unexpectedly, processes to prepare the catalyst compositions as disclosed above can result in oligomerization activity exclusively, i.e., processes to prepare catalyst compositions disclosed herein can exclude polymerization activity of the transition metal amine bis(phenolate) compounds and activator-support, to produce desirable oligomer products with vinylidene end groups in high selectivity.

In certain aspects, the catalyst compositions can be prepared by first contacting the activator-support and the organoaluminum compound to form a first mixture, and thereafter contacting the first mixture and the transition metal amine bis(phenolate) catalyst to form the catalyst composition. In certain aspects, the catalyst composition can be contacted with a reactant (e.g., monomer) immediately (e.g., within 1 second, within 5 seconds, within 15 seconds, within 1 minute, within 5 minutes) after contacting the first mixture and the transition metal amine bis(phenolate) catalyst.

In certain aspects, the transition metal amine bis(phenolate) catalyst can be contacted with the first mixture within an oligomerization reactor, thereby forming the catalyst composition in situ in the presence of an olefin reactant and diluent. Thus, contacting the first mixture and the transition metal amine bis(phenolate) catalyst can be conducted in concert with the formation of oligomer product as discussed further below. In such aspects, an amount of the first mixture can be maintained within the oligomerization reactor, and the transition metal amine bis(phenolate) can be transferred to the oligomerization reactor independently from the first mixture. In certain aspects, the transition metal amine bis(phenolate) can be transferred to the oligomerization reactor independently, or in solution. Alternatively, the transition metal amine bis(phenolate) compound can be transferred into the reactor within a reactant mixture, comprising an olefin and optionally a diluent as described above.

Conditions for each of the contacting steps are limited only as necessary to preserve or promote the catalytic activity of the resulting catalyst composition. In certain aspects, one or both contacting steps can be performed in an inert atmosphere, e.g., N₂ gas. In certain aspects, the activator-support and the organoaluminum compound can be contacted in the presence of a diluent. Suitable diluents can include, but are not limited to, the monomer being oligomerized and hydrocarbons that are liquids under reaction conditions. Examples of suitable diluents include, but are not limited to, hydrocarbons such as propane, cyclohexane, isobutane, n-butane, n-pentane, isopentane, neopentane, and n-hexane, heptanes, octanes, petroleum ether, light naphtha, heavy naphtha, or any combination thereof.

### Oligomerization Processes

Oligomerization processes incorporating catalyst compositions disclosed above can produce highly selective oligomer products. Generally, oligomerization processes disclosed herein can comprise contacting any catalyst composition disclosed herein with an alpha olefin monomer and optionally H₂ in an oligomerization reactor, under oligomerization conditions, and forming an oligomer product in the oligomerization reactor.

A wide range of alpha olefin monomers can be reacted in the oligomerization processes provided herein. For example, the alpha olefin can comprise, consist essentially of, or consist of, a C₄ to C₃₀ alpha olefin; alternatively, a C₄ to C₁₈ alpha olefin; alternatively, a C₄ to C₁₄ alpha olefin; alternatively, a C₅ to Cis alpha olefin; alternatively, a C₆ to C₁₆ alpha olefin; or alternatively, a C₈ to C₁₂ alpha olefin. In an aspect, the oligomer product can be produced from an alpha olefin comprising, consisting essentially of, or consisting of, a C₆ alpha olefin, a C₈ alpha olefin, a C₁₀ alpha olefin, a C₁₂ alpha olefin, a C₁₄ alpha olefin, a C₁₆ alpha olefin, or any combination thereof, alternatively, a C₈ alpha olefin, a C₁₀ alpha olefin, a C₁₂ alpha olefin, or any combination thereof, alternatively, a C₆ alpha olefin; alternatively, a C₈ alpha olefin; alternatively, a C₁₀ alpha olefin; alternatively, a C₁₂ alpha olefin; alternatively, a C₁₄ alpha olefin; alternatively, a C₁₆ alpha olefin; or alternatively, a C₁₈ alpha olefin. In a further aspect, the alpha olefin can comprise, consist essentially of, or consist of 1-butene, 1-pentene, 1-hexene, 1-octene, 1-decene, 1-dodecene, 1-tetradecene, 1-hexadecene, or any combination thereof. For instance, the alpha olefin can comprise, consist essentially of, or consist of 1-octene; alternatively, 1-decene; or alternatively, 1-dodecene.

Any suitable amount of the alpha olefin feed can be a normal alpha olefin. Generally, the alpha olefin contains at least 50 wt. % normal alpha olefin(s), and more often, contains at least 60 wt. %, at least 70 wt. %, at least 80 wt. %, at least 85 wt. %, at least 90 wt. %, at least 92.5 wt. %, or at least 95 wt. % of normal alpha olefin(s), and in some aspects, less than or equal to 99.9 wt. %, less than or equal to 99.5 wt. %, less than or equal to 97 wt. %, or less than or equal to 95 wt. % of normal alpha olefin(s), and in other aspects, a range from any minimum amount disclosed herein to any maximum amount disclosed herein of normal alpha olefin(s), e.g., the alpha olefin contains from 85 wt. % to 95 wt. % or from 90 wt. % to 99 wt. %, of 1-hexene, 1-octene, 1-decene, 1-dodecene, or 1-tetradecene. Thus, mixtures of various alpha olefins (or normal alpha olefins) having different numbers of carbon atoms can be used, or alpha olefins (or normal alpha olefins) having predominantly a single number of carbon atoms can be used.

Certain ratios of components may be used to control the oligomerization process. For instance, increasing the weight ratio of the catalyst compound in the catalyst composition to the alpha olefin monomer can lead to a higher conversion, but may also lead to less of the desirable dimer and trimer products. Nonetheless, the catalyst composition and alpha olefin monomer can be contacted at a weight ratio of the catalyst compound to the alpha olefin monomer ranging from 10:1 to 1:10,000, from 1:1 to 1:5,000, from 1:10 to 1:1,000, or from 1:100 to 1:500.

Oligomerization conditions utilized in the oligomerization processes can comprise an oligomerization temperature from −10° C. to 250° C., from 20° C. to 180° C., or from 50° C. to 160° C.; alternatively, from 55° C. to 160° C.; alternatively, from 60° C. to 155° C.; alternatively, from 65° C. to 150° C.; alternatively, from 70° C. to 140° C.; or alternatively, from 75° C. to 140° C. In another non-limiting aspect, the oligomerization temperature can range from 70° C. to 90° C.; alternatively, from 90° C. to 120° C.; or alternatively, from 110° C. to 140° C.

In another non-limiting aspect, the oligomerization conditions utilized in the oligomerization processes disclosed herein can comprise performing the oligomerization reaction in the presence of hydrogen. The hydrogen partial pressure in the oligomerization reaction can be any pressure of hydrogen that does not adversely affect the oligomerization reaction. In some non-limiting aspects, the oligomerization conditions can include a partial pressure of hydrogen at least 0.1 psig and often up to and including a partial pressure of 50 psig. Typical ranges for the hydrogen partial pressure can include from 0.1 psig to 50 psig, from 0.1 psig to 20 psig, from 0.1 psig to 10 psig, from 1 psig to 20 psig, from 1 psig to 10 psig, from 2 psig to 20 psig, or from 2 psig to 10 psig.

The oligomer product often can contain a dimer of the alpha olefin monomer and a trimer of the alpha olefin monomer. Advantageously, the disclosed oligomerization processes can produce oligomer products having a relatively high amount of dimers and trimers that can be useful in subsequent reactions and in the production of polyalphaolefins, and a relatively low amount of tetramer and heavier oligomer.

The oligomer product formed by the oligomerization processes, therefore, can be characterized by the relative amount of specific oligomers. In certain aspects, the oligomer product can comprise less than or equal to 10 wt. %, less than or equal to 5 wt. %, less than or equal to 2 wt. %, less than or equal to 1 wt. %, or less than or equal to 0.5 wt. % tetramer. Additionally, or alternatively, the oligomer product can comprise at least 60 wt. %, at least 70 wt. %, at least 80 wt. %, at least 85 wt. %, at least 90 wt. %, or at least 95 wt. % of dimer and trimer (total). In certain aspects, and beneficially, the dimer may be the majority component of the oligomer product, and the oligomer product can contain at least 30 wt. %, at least 40 wt. %, at least 50 wt. %, at least 55 wt. %, at least 60 wt. %, at least 65 wt. %, at least 70 wt. %, or at least 75 wt. % alpha olefin dimer, based on total oligomers in the oligomer product, and excluding unreacted alpha olefin monomer. Unreacted alpha olefin monomer is excluded from the compositional breakdown of the oligomer product.

Oligomers comprising vinylidene end groups can be desirable for their high reactivity relative to internal and branched dimers and trimers of the alpha olefin monomer. In certain aspects, the dimer and/or trimer in the oligomer product comprises at least 50 wt. %, at least 60 wt. %, at least 70 wt. %, at least 75 wt. %, at least 80 wt. %, at least 85 wt. %, at least 90 wt. %, or at least 95 wt. % vinylidene. It follows then, that the amount of internal olefin within the dimer and/or trimer portion of the oligomer product generally can be less than or equal to 15 wt. %, less than or equal to 12 wt. %, or less than or equal to 10 wt. %.

The conversion of alpha olefin monomer in the oligomerization reactor is not limited to a particular amount, however, generally the minimum alpha olefin monomer conversion can be at least at least 40, 50, 60, or 65%, while the maximum alpha olefin monomer conversion can be 99, 95, 90, 80, 75, or 70%. Generally, the alpha olefin monomer conversion in the reactor can range from any minimum conversion to any maximum conversion described herein. For instance, the alpha olefin monomer conversion can range from 40 to 99%, from 50 to 95%, from 60 to 90%, from 60 to 80%, from 65 to 80%, from 65 to 75%, or from 65 to 70%. The alpha olefin monomer conversion is based on the amount of alpha olefin monomer entering the reactor and the amount of (unreacted) alpha olefin monomer in the effluent stream.

The oligomerization processes, in certain aspects, can further comprise a step of separating at least a portion of the catalyst composition from the oligomer product using any suitable technique, e.g., by filtration. Likewise, the oligomerization processes can further comprise a step of separating unreacted alpha olefin monomer from the oligomer product using any suitable technique, e.g., wiped film evaporating, distillation, short path distillation, or any combination thereof. Optionally, the oligomerization processes can further comprise recycling either or both of the recovered catalyst composition and the recovered unreacted alpha olefin monomer, for instance, for re-use in the oligomerization processes.

Further still, the oligomerization processes, in certain aspects, can comprise a step of fractionating the oligomer product into alpha olefin dimer, alpha olefin trimer, and alpha olefin heavies (including alpha olefin tetramer and higher oligomers), using any suitable technique, e.g., wiped film evaporating, distillation, short path distillation, or any combination thereof. The process of fractionating an oligomer product into several oligomer fractions is generally known and techniques and conditions for conducting the fractionation, and subsequent separation, and/or purification steps will be understood by those of skill in the art.

### EXAMPLES

The invention is further illustrated by the following examples, which are not to be construed in any way as imposing limitations to the scope of this invention. Various other aspects, modifications, and equivalents thereof which, after reading the description herein, can suggest themselves to one of ordinary skill in the art without departing from the spirit of the present invention or the scope of the appended claims.

### Examples 1-5

Oligomerizations reactions were performed generally as follows, and as described for individual Examples 1-5 below. Generally, for each of Examples 1-5, the appropriate catalyst mixture was prepared as described for each example and combined with 6 mL monomer (1-decene or 2-octene, as appropriate) under a nitrogen purge. The reaction mixture was stirred for 1 hour at a temperature of 130° C., after which the reaction mixture was cooled to 35° C. A solution of 10% HCl in isopropyl alcohol (10 mL total charge) was added to quench the oligomerization reaction and the reactor contents were removed. The reaction mixture was then filtered and analyzed by gas chromatography to determine the yield of oligomer product formed in the reaction mixture, and to determine the relative amount of certain oligomers within the oligomer product (e.g., the dimer:trimer weight ratio, vinylidene isomers).

Gas chromatographic (GC) analyses were performed using a split injection method on a Bruker 430-GC gas chromatograph with a flame ionization detector (FID). Initial oven temperature was 70° C. for 2 minutes and increased 5° C./min to 290° C. and held for 7 minutes. The column was an all-purpose capillary column (Agilent J&W VF-5 ms, 30 m×0.25 mm×0.25 m). Data analysis was performed using CompassCDS software. The distribution of olefin end groups was determined using ¹H NMR on a Bruker 300 MHz NMR.

In each of Examples 1-5, the activator-support (A-S) was a fluorided silica-coated alumina (60:40 alumina:silica by weight) containing 4 wt. % F and having a d50 average particle size of 35 microns, a BET surface area of 450 m²/g, and a pore volume of 1.1 mL/g. The organoaluminum in each example was triisobutylaluminum (TIBA). The structure of the amine bis(phenolate) catalyst used in Examples 1-5 is shown below.

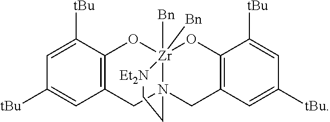

Results of the oligomerization reactions are summarize in Table I. Notably, for each of Examples 1-5, no tetramers or higher oligomers were observed in the reaction mixture by gas chromatography.

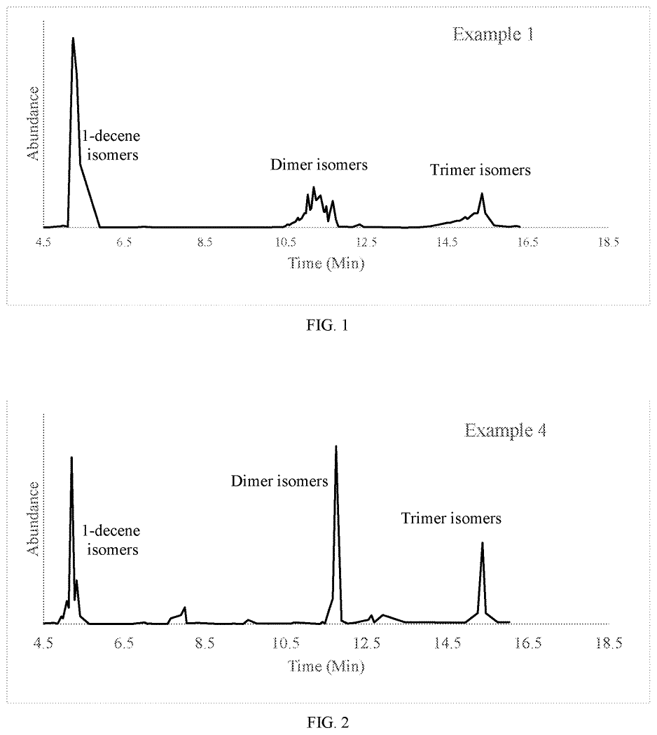

TABLE I
Comparison of Oligomer Properties.

| | | | | | Catalyst | | | |
|---|---|---|---|---|---|---|---|---|
| | A-S | TiBA | | | Activity | Dimer | Trimer | Vinylidene |
| Ex. | (g) | (mmol) | Catalyst (g) | Monomer | (g/g/hr) | (wt. %) | (wt. %) | (wt. %) |
| 1 | 1.5 | 0 | 0.01 | 1-decene | 356 | 56 | 44 | (see FIG. 1) |
| 2 | 1.5 | 0 | 0 | 1-decene | 0 | 57 | 43 | |
| 3 | 1.5 | 1.2 | 0 | 1-decene | 0 | 0 | 0 | |
| 4 | 1.5 | 1.2 | 0.01 | 1-decene | 333 | 60 | 40 | 99% |
| 5 | 1.5 | 1.2 | 0.01 | 2-octene | 0 | 0 | 0 | |

In Example 1, a catalyst mixture was prepared under an N₂ atmosphere by combining the activator-support (A-S) and the transition metal amine bis(phenolate) complex. No organoaluminum was added to the catalyst mixture. The catalyst mixture was then added to the reaction vessel containing 1-decene. As shown in Table I, the reaction produced an oligomer product with 80% conversion of the monomer, in a roughly 60:40 split of dimer:trimer. FIG. 1 presents a gas chromatograph of the resulting products, which show that both dimer and trimer within the oligomer product were a mixture of many different isomers.

Example 2 was conducted without organoaluminum in the catalyst mixture (as for Example 1), and also without the transition metal amine bis(phenolate) catalyst. Thus, the catalyst mixture consisted of only the activator-support. Surprisingly, the monomer conversion, and dimer:trimer ratio of the product mixture was nearly identical to that observed in Example 1, producing a monomer conversion of 81%, and a dimer:trimer ratio of 57:43, even in the absence of the catalyst. Thus, the results of Example 2 demonstrate that the oligomerization activity observed in Example 1 was not dependent on the presence of the transition metal amine bis(phenolate) complex.

Example 3 was conducted under the same conditions as Examples 1-2, with the exception that only the transition metal amine bis(phenolate) complex was omitted. TIBA was added to a mixture of the activator-support in 4 mL hexanes, and the mixture was introduced to the reactor with 1-decene (6 mL). The result was a complete lack of oligomerization activity, as noted by the monomer conversion of 0%. Thus, whereas the activity from Example 2 could be attributed to the presence of the activator-support (which generated a mixture of many different oligomer isomers), the addition of TIBA prevented this oligomerization activity.

Example 4 was conducted by varying the conditions of Example 3 above to include the amine bis(phenolate) catalyst within the catalyst mixture. In Example 4, TIBA was added to a mixture of the activator-support in hexanes (4 mL). A solution of the amine bis(phenolate) catalyst and neat monomer (1-decene, 6 mL) was prepared separately, and added to the mixture of TIBA and activator-support in hexanes. Oligomerization activity was observed with a monomer conversion of 74%. Surprisingly, the monomer conversion was near that of the prior Examples 1-2, despite the presence of organoaluminum presumably suppressing activity resulting from the presence of the activator-support, as demonstrated in Example 3. Also surprising, the dimer:trimer ratio was slightly improved to 60:40 relative to the oligomer product of Examples 1-2.

Perhaps most unexpectedly, the undesired dimer and trimer isomers observed in the products of Examples 1-2 (see FIG. 1) were almost completely eliminated by premixing the organoaluminum and activator-support prior to combining the mixture with the amine bis(phenolate) catalyst pre-mixed with monomer. FIG. 2 presents a gas chromatograph of the oligomer product obtained in Example 4. As shown, single isomers of dimer and trimer were present as the overwhelming majority of the oligomer product, representing more than 99% of the dimer and trimer product, each of which was the desirable vinylidene isomer.

The incredible vinylidene selectivity of the catalyst system of Example 4 was confirmed by Example 5, where the same oligomerization conditions were employed to the oligomerization of 2-octene, an internal olefin. No conversion of the internal olefin monomer was observed, indicating that the catalyst system of Example 4 exhibited no reactivity to internal olefins.

### ASPECTS

The invention is described above with reference to numerous aspects and specific examples. Many variations will suggest themselves to those skilled in the art in light of the above detailed description. All such obvious variations are within the full intended scope of the appended claims. Other aspects of the invention can include, but are not limited to, the following (aspects are described as “comprising” but, alternatively, can “consist essentially of” or “consist of”):

Aspect 1. A catalyst composition comprising:
a catalyst having Formula (I):

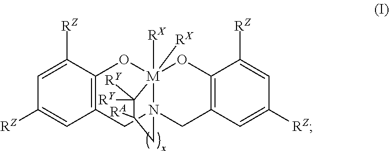

wherein:
   M is Zr or Hf;
   each R^{X} independently is a halogen or a C₁ to C₁₈ hydrocarbyl group;
   each R^{Y} independently is a C₂ to C₁₂ hydrocarbyl group; and
   each R^{Z} independently is H or a C₁ to C₁₂ hydrocarbyl group;
   x is in a range from 1 or 2, or alternatively, x is in a range from 3 to 6; and
   R^{A} is H or joined with an R group to form an aromatic heterocyclic group; and an activator-support.

Aspect 2. The catalyst composition defined in aspect 1, further comprising an organoaluminum compound (e.g., an alkylaluminum).

Aspect 3. The catalyst composition defined in aspect 2, wherein the organoaluminum compound comprises trimethylaluminum (TMA), triethylaluminum (TEA), tri-n-propylaluminum (TNPA), tri-n-butylaluminum (TNBA), triisobutylaluminum (TIBA), tri-n-hexylaluminum, tri-n-octylaluminum (TNOA), or combinations thereof.

Aspect 4. The catalyst composition defined in aspect 2 or 3, wherein the organoaluminum compound comprises triethylaluminum (TEA), tri-n-butylaluminum (TNBA), triisobutylaluminum (TIBA), or a combination thereof.

Aspect 5. The catalyst composition defined in any one of aspects 1-4, wherein M is Zr.

Aspect 6. The catalyst composition defined in any one of aspects 1-5, wherein each R^{X} independently is a C₁ to C₈ hydrocarbyl group.

Aspect 7. The catalyst composition defined in any one of aspects 1-6, wherein each R^{X} independently is a methyl group, an ethyl group, a propyl group, a butyl group (e.g., n-butyl, iso-butyl), a pentyl group (e.g., cyclopentyl), a hexyl group, a phenyl, or a benzyl group.

Aspect 8. The catalyst composition defined in any one of aspects 1-7, wherein each R^{X} is the same.

Aspect 9. The catalyst composition defined in any one of aspects 1-8, wherein each R^{X} is a benzyl group.

Aspect 10. The catalyst composition defined in any one of aspects 1-5, wherein each R^{X} is Cl.

Aspect 11. The catalyst composition defined in any one of aspects 1-10, wherein each R^{Y} independently is an aliphatic C₂ to C₈ hydrocarbyl group.

Aspect 12. The catalyst composition defined in any one of aspects 1-11, wherein each R^{Y} is an ethyl group.

Aspect 13. The catalyst composition defined in any one of aspects 1-11, wherein each R^{Z} is different.

Aspect 14. The catalyst composition defined in any one of aspects 1-13, wherein each R^{Z} independently is a methyl group, an ethyl group, a propyl group (e.g., n-propyl, isopropyl, cyclopropyl), a butyl group (e.g., n-butyl, iso-butyl, cyclobutyl), a pentyl group, a hexyl group, a phenyl group, or a benzyl group.

Aspect 15. The catalyst composition defined in any one of aspects 1-14, wherein each R^{Z} is the same (e.g., each R^{Z} is a t-butyl group).

Aspect 16. The catalyst composition defined in any one of aspects 1-9, wherein the catalyst has the formula:

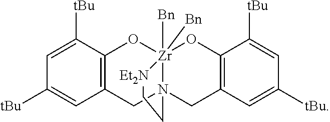

Aspect 17. The catalyst composition defined in any one of aspects 1-16, wherein the activator-support comprises any solid oxide treated with any electron-withdrawing anion disclosed herein.

Aspect 18. The catalyst composition defined in an one of aspects 1-17, wherein the activator-support comprises fluorided alumina, chlorided alumina, bromided alumina, sulfated alumina, fluorided silica-alumina, chlorided silica-alumina, bromided silica-alumina, sulfated silica-alumina, fluorided silica-zirconia, chlorided silica-zirconia, bromided silica-zirconia, sulfated silica-zirconia, fluorided silica-titania, fluorided silica-coated alumina, fluorided-chlorided silica-coated alumina, sulfated silica-coated alumina, phosphated silica-coated alumina, or any combination thereof.

Aspect 19. The catalyst composition defined in any one of aspects 1-18, wherein the activator-support comprises fluorided alumina, sulfated alumina, fluorided silica-alumina, sulfated silica-alumina, fluorided silica-coated alumina, fluorided-chlorided silica-coated alumina, sulfated silica-coated alumina, or any combination thereof.

Aspect 20. The catalyst composition defined in any one of aspects 1-19, wherein the activator-support comprises a fluorided solid oxide and/or a sulfated solid oxide.

Aspect 21. The catalyst composition defined in any one of aspects 1-20, wherein the activator-support comprises fluorided silica-coated alumina, sulfated alumina, or a combination thereof.

Aspect 22. The catalyst composition defined in any one of aspects 2-21, wherein a weight ratio of the organoaluminum compound to the activator-support in the catalyst composition is in any range disclosed herein, e.g., from 10:1 to 1:100, from 10:1 to 1:10, or from 1:1 to 1:10.

Aspect 23. The catalyst composition defined in any one of aspects 2-21, wherein a weight ratio of the organoaluminum compound to the catalyst in the catalyst composition is in any range disclosed herein, e.g., from 100:1 to 1:10, or from 50:1 to 1:1.

Aspect 24. The catalyst composition defined in any one of aspects 1-23, wherein the catalyst composition is substantially free of aluminoxane compounds, organoboron or organoborate compounds, ionizing ionic compounds, or combinations thereof.

Aspect 25. The catalyst composition defined in any one of aspects 1-23, further comprising an aluminoxane compound, an organoboron or organoborate compound, an ionizing ionic compound, an organozinc compound, an organomagnesium compound, an organolithium compound, or any combination thereof.

Aspect 26. A process to prepare the catalyst composition defined in any one of aspects 2-25, the process comprising:
contacting the activator-support and the organoaluminum compound to form a first mixture; and
contacting the first mixture and the catalyst to form the catalyst composition.

Aspect 27. An oligomerization process comprising:
(i) contacting the catalyst composition defined in any one of aspects 2-25 (or the catalyst composition produced by the process defined in aspect 26), a C₄ to C₃₀ alpha olefin monomer, and optionally hydrogen, in an oligomerization reactor under oligomerization conditions; and
(ii) forming an oligomer product in the oligomerization reactor.

Aspect 28. The process defined in aspect 27, wherein the alpha olefin monomer comprises any C₄ to C₁₄ alpha olefin or C₈ to C₁₂ alpha olefin disclosed herein, e.g., 1-octene and/or 1-decene.

Aspect 29. The process defined in aspect 27 or 28, wherein the alpha olefin monomer comprises a branched alpha olefin.

Aspect 30. The process defined in any one of aspects 27-29, wherein the alpha olefin monomer comprises a mixture of alpha olefins (e.g., a mixture of C₈ to C₁₂ alpha olefins, or a mixture of C₁₀ alpha olefins).

Aspect 31. The process defined in any one of aspects 27-30, wherein a weight ratio of the catalyst to the alpha olefin monomer is in any range disclosed herein, e.g., from 10:1 to 1:10,000, from 1:1 to 1:5,000, from 1:10 to 1:1,000, or from 1:100 to 1:500.

Aspect 32. The process defined in any one of aspects 27-31, wherein an amount of the organoaluminum compound in the oligomerization reactor is in a range from 1,000 ppmw to 100,000 ppmw, based on alpha olefin monomer.

Aspect 33. The process defined in any one of aspects 27-32, wherein an amount of the catalyst in the oligomerization reactor is in a range from 50 ppmw to 10,000 ppmw, based on alpha olefin monomer.

Aspect 34. The process defined in any one of aspects 27-33, wherein the oligomerization conditions comprise an oligomerization temperature in any range disclosed herein, e.g., from −10° C. to 250° C., from 20° C. to 180° C., from 50° C. to 160° C., or from 70° C. to 140° C.

Aspect 35. The process defined in any one of aspects 27-34, wherein the catalyst composition is contacted with the alpha olefin monomer and H₂ at any suitable hydrogen partial pressure (e.g., from 0.1 to 10 psig of H₂).

Aspect 36. The process defined in any one of aspects 27-34, wherein no hydrogen is added to the oligomerization reactor.

Aspect 37. The process defined in any one of aspects 27-36, wherein the oligomer product comprises any amount of tetramer disclosed herein, e.g., less than or equal to 10 wt. %, less than or equal to 5 wt. %, less than or equal to 3 wt. %, less than or equal to 2 wt. %, or less than or equal to 1 wt. %.

Aspect 38. The process defined in any one of aspects 27-37, wherein the oligomer product comprises any amount of alpha olefin dimer and alpha olefin trimer disclosed herein, e.g., at least 60 wt. %, at least 70 wt. %, at least 80 wt. %, at least 85 wt. %, at least 90 wt. %, or at least 95 wt. %.

Aspect 39. The process defined in any one of aspects 27-38, wherein the oligomer product comprises any amount of alpha olefin dimer disclosed herein, e.g., at least 30 wt. %, at least 40 wt. %, at least 50 wt. %, at least 55 wt. %, at least 60 wt. %, at least 65 wt. %, at least 70 wt. %, or at least 75 wt. %.

Aspect 40. The process defined in any one of aspects 27-39, wherein the oligomer product comprises more vinylidene-terminated oligomers than that of an otherwise identical process in which the activator-support is not contacted with an organoaluminum compound prior to the catalyst.

Aspect 41. The process defined in any one of aspects 27-40, wherein a conversion of the alpha olefin monomer is in any range disclosed herein, e.g., at least 50%, at least 60%, at least 70%, at least 75%, or at least 80%.

Aspect 42. The process defined in any one of aspects 27-41, wherein a conversion of the alpha olefin monomer is within 20%, within 10%, within 5% of an otherwise identical process in which the catalyst composition does not comprise the catalyst and/or the organoaluminum compound.

Aspect 43. The process defined in any one of aspects 38-42, wherein the alpha olefin dimer comprises any amount of vinylidene disclosed herein, e.g., at least 95 wt. %, at least 98 wt. %, at least 80 wt. %, or at least 85 wt. %.

Aspect 44. The process defined in any one of aspects 27-43, further comprising a step of separating at least a portion of the catalyst composition from the oligomer product using any technique disclosed herein, e.g., filtration.

Aspect 45. The process defined in aspect 44, further comprising recycling the separated catalyst composition.

Aspect 46. The process defined in any one of aspects 27-45, further comprising a step of separating unreacted alpha olefin monomer from the oligomer product using any technique disclosed herein, e.g., wiped film evaporating, distillation, short path distillation, or any combination thereof.

Aspect 47. The process defined in aspect 46, further comprising recycling the unreacted alpha olefin monomer.

Aspect 48. The process defined in any one of aspects 27-47, further comprising a step of fractionating the oligomer product into alpha olefin dimer, alpha olefin trimer, and alpha olefin heavies including alpha olefin tetramer and higher oligomers, using any technique disclosed herein, e.g., wiped film evaporating, distillation, short path distillation, or any combination thereof.

Aspect 49. The process defined in any one of aspects 27-48, wherein contacting the first mixture and the catalyst to form the catalyst composition is conducted within the oligomerization reactor.

What is claimed is:

1. An oligomerization process comprising:
(i) contacting a catalyst composition, a C₄ to C₃₀ alpha olefin monomer, and optionally hydrogen, in an oligomerization reactor under oligomerization conditions; and
(ii) forming an oligomer product in the oligomerization reactor;
wherein the catalyst composition comprises:
a catalyst having Formula (I): 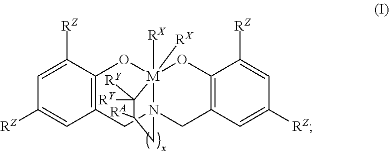
wherein:
M is Zr or Hf;
each R^{X} independently is a halogen or a C₁ to C₁₈ hydrocarbyl group;
each R^{Y} independently is a C₂ to C₁₂ hydrocarbyl group; and
each R^{Z} independently is H or a C₁ to C₁₂ hydrocarbyl group;
x is 1, 2, or 3; and
R^{A} is H or joined with an R group to form an aromatic heterocyclic group;
and
an activator-support.

2. The process of claim 1, wherein the catalyst has the formula:

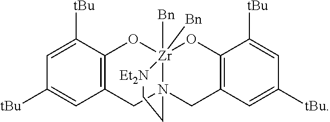

3. The process of claim 1, wherein the catalyst composition further comprises an organoaluminum compound.

4. The process of claim 3, wherein the organoaluminum compound comprises triethylaluminum (TEA), tri-n-butylaluminum (TNBA), triisobutylaluminum (TIBA), or a combination thereof.

5. The process of claim 3, wherein a weight ratio of the organoaluminum compound to the activator-support in the catalyst composition is in a range from 1:1 to 1:10.

6. The process of claim 3, wherein a weight ratio of the organoaluminum compound to the catalyst in the catalyst composition is in a range from 1:1 to 50:1.

7. The process of claim 3, wherein a conversion of the alpha olefin monomer is within 20% of an otherwise identical process in which the catalyst composition does not comprise the organoaluminum compound.

8. The process of claim 3, wherein the process further comprises before step (i):
contacting the activator-support and the organoaluminum compound to form a first mixture; and
contacting the first mixture and the catalyst having formula (I) to form the catalyst composition.

9. The process of claim 8, wherein contacting the first mixture and the catalyst to form the catalyst composition is conducted within the oligomerization reactor.

10. The process of claim 1, wherein the activator-support comprises fluorided alumina, chlorided alumina, bromided alumina, sulfated alumina, fluorided silica-alumina, chlorided silica-alumina, bromided silica-alumina, sulfated silica-alumina, fluorided silica-zirconia, chlorided silica-zirconia, bromided silica-zirconia, sulfated silica-zirconia, fluorided silica-titania, fluorided silica-coated alumina, fluorided-chlorided silica-coated alumina, sulfated silica-coated alumina, phosphated silica-coated alumina, or any combination thereof.

11. The process of claim 1, wherein the activator-support comprises fluorided silica-coated alumina, sulfated alumina, or a combination thereof.

12. The process of claim 1, wherein the alpha olefin monomer comprises a C₄ to C₁₄ alpha olefin.

13. The process of claim 1, wherein the alpha olefin monomer comprises 1-decene.

14. The process of claim 1, wherein the oligomer product comprises:
less than or equal to 10 wt. % of alpha olefin tetramer;
at least 90 wt. % of alpha olefin dimer and alpha olefin trimer;
at least 55 wt. % of alpha olefin dimer;
alpha olefin dimer comprising at least 98 wt. % of vinylidene; or
any combination thereof.

15. The process of claim 1, wherein a conversion of the alpha olefin monomer is within 20% of an otherwise identical process in which the catalyst composition does not comprise the catalyst.

16. The process of claim 1, further comprising:
separating at least a portion of the catalyst composition from the oligomer product;
fractionating the oligomer product into alpha olefin dimer and alpha olefin trimer; or
both.

17. The process of claim 1, wherein:
the oligomer product comprises at least 90 wt. % of alpha olefin dimer and alpha olefin trimer;
the alpha olefin dimer comprises at least 90 wt. % of vinylidene; and
the catalyst has the formula:

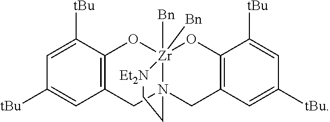

18. A catalyst composition comprising:
a catalyst having Formula (I): 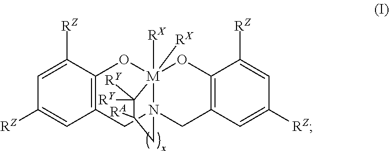
wherein:
M is Zr or Hf;
each R^{X} independently is a halogen or a C₁ to C₁₈ hydrocarbyl group;
each R^{Y} independently is a C₂ to C₁₂ hydrocarbyl group; and
each R^{Z} independently is H or a C₁ to C₁₂ hydrocarbyl group;
x is 1, 2, or 3; and
R^{A} is H or joined with an R group to form an aromatic heterocyclic group;
and
an activator-support.

19. The catalyst composition of claim 18, wherein:
the catalyst composition further comprises an organoaluminum compound; and
the catalyst has the formula:

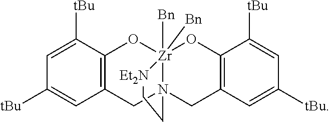

20. A process to prepare an oligomerization catalyst composition, the process comprising:
contacting an activator-support and an organoaluminum compound to form a first mixture; and
contacting the first mixture and a transition metal amine bis(phenolate) complex to form the oligomerization catalyst composition; wherein:
the activator-support comprises fluorided silica-coated alumina, sulfated alumina, or a combination thereof;
the organoaluminum compound comprises triethylaluminum (TEA), tri-n-butylaluminum (TNBA), triisobutylaluminum (TIBA), or a combination thereof; and
the transition metal amine bis(phenolate) complex has Formula (I): 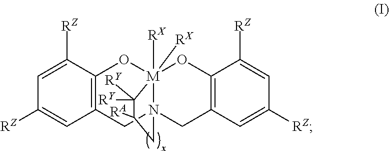
wherein:
M is Zr or Hf;
each R^{X} independently is a halogen or a C₁ to C₁₈ hydrocarbyl group;
each R^{Y} independently is a C₂ to C₁₂ hydrocarbyl group; and
each R^{Z} independently is H or a C₁ to C₁₂ hydrocarbyl group;
x is 1, 2, or 3; and
R^{A} is H or joined with an R group to form an aromatic heterocyclic group.
